(19) United States
(12) Patent Application Publication
(10) Pub. No.: US 2007/0197837 A1
(43) Pub. Date: Aug. 23, 2007

(54) Method for the hydrodecomposition of ammonium formates in polyolcontaining reaction mixtures

(75) Inventors: Michael Koch, Mannheim (DE); Alexander Wartini, Heidelberg (DE); Steffen Maas, Bubenheim (DE); Tilman Sirch, Schifferstadt (DE); Matthias Dernbach, Dossenheim (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft

(21) Appl. No.: 10/566,374

(22) PCT Filed: Jul. 7, 2004
(86) PCT No.: PCT/EP04/07396

§ 371 Date: Nov. 21, 2006

(51) Int. Cl. C07C 43/32

(52) U.S. Cl. 568/594

(57) ABSTRACT

A process for removing trialkylammonium formate from methylolalkanes which have been obtained by condensation of formaldehyde with a higher aldehyde comprises decomposing trialkylammonium formate at elevated temperature in the presence of a hydrogen-containing gas over a catalyst comprising ruthenium supported on titanium dioxide. The process makes it possible to separate off the trialkylammonium formate from methylolalkanes prepared by the organic Cannizzaro process or by the hydrogenation process.

The invention relates to the field of industrial organic chemistry. More precisely, the present invention provides a process for the effective hydrogenating decomposition of trialkylammonium formate which is present in methylolalkanes and has been formed from the trialkylamine used as catalyst in the preparation of the methylolalkanal and the formic acid formed as by-product.

The condensation of formaldehyde with CH-acid higher alkanals to form methylol-alkanals, in general dimethylolalkanals and trimethylolalkanals, and conversion of the compounds obtained into polyols is a widely employed process in industrial chemistry. Examples of important triols obtained in this way are trimethylolpropane, trimethylol-ethane and trimethylolbutane, which have found widespread use in the production of surface coatings, urethanes and polyesters. Further important compounds are pentaerythritol, obtainable by condensation of formaldehyde and acetaldehyde, and also neopentyl glycol from isobutyraladehyde and formaldehyde. The tetravalent alcohol pentaerythritol is likewise frequently used in the surface coatings industry, but has also achieved great importance in the production of explosives.

The polyols mentioned can be prepared by various methods. One method is the Cannizzaro process which is further subdivided into the inorganic Cannizzaro process and the organic Cannizzaro process. In the inorganic variant, an excess of formaldehyde is reacted with the corresponding alkanal in the presence of stoichiometric amounts of an inorganic base such as NaOH or Ca(OH)₂. The methylolalkanal formed in the first step reacts in the second step with the excess formaldehyde in a disproportionation reaction to form the corresponding polyol and the formate of the respective base, i.e., for example, sodium or calcium formate.

In the organic Cannizzaro process, a tertiary amine, generally a trialkylamine, is used in place of the inorganic base. The reaction proceeds as described above, with one equivalent of the ammonium formate of the corresponding amine being formed. This can be worked up further by appropriate methods, so that at least the amine can be recovered and return to the reaction. The crude polyol obtained can be worked up in various ways to give the pure polyol.

A further development is the hydrogenation process in which an appropriate alkanal and formaldehyde are reacted with one another not in the presence of at least stoichiometric amounts but of catalytic amounts of a tertiary amine, generally from about 5 to 10 mol %. In this process, the reaction stops at the stage of 2,2-dimethylol-alkanal which is subsequently converted into trimethylolalkane by hydrogenation. A description of the effective process may be found in WO 98/28253 of the present applicant.

A number of variants of this hydrogenation process are described, inter alia, in the patent applications DE-A-25 07 461, DE-A-27 02 582, DE-A-28 13 201 and DE-A-33 40 791.

Although the hydrogenation process advantageously does not form stoichiometric amounts of the formate as in the organic Cannizzaro process, trialkylammonium formate is formed as product of a cross-Cannizzaro reaction occurring to a small extent as secondary reaction.

Trialkylammonium formates react under particular conditions, for example, the dewatering or heating of trimethylolalkane solutions obtained, to form trialkylamine and trimethylolpropane formate. These decrease the yield of trimethylolalkane and are difficult to dissociate without undesirable degradation reactions. There is therefore particular interest in the removal of trialkylammonium formates.

DE 198 48 569 discloses a process for the decomposition of formates of tertiary amines which are present as by-products in trimethylolalkane solutions prepared by the organic Cannizzaro process. These formates are decomposed by heating, preferably in the presence of modified noble metal catalysts and under superatmospheric pressure, into hydrogen and carbon dioxide and/or water and carbon monoxide and the tertiary amine. The formate conversions in this process are unsatisfactory, and the formation of further by-products is also observed.

DE 101 52 525 discloses the decomposition of trialkylammonium formates over heterogeneous catalysts comprising at least one metal of groups 8 to 12 of the Periodic Table, with particular preference being given to supported copper-, nickel- and/or cobalt-containing catalysts.

In addition, the abovementioned process has only limited suitability for the effective work-up of a trimethylolalkane mixture obtained by the hydrogenation process in which only catalytic amounts of trialkylamine are used and the product mixture thus also contains only small amounts of trialkyammonium formate.

It is an object of the present invention to provide a process which is suitable for the work-up of reaction mixtures obtained by the hydrogenation process and also those obtained by the organic Cannizzaro process. Furthermore, this process should make it possible to decompose trialkylammonium formates with higher conversions than have been possible using the processes known from the prior art. In addition, this decomposition should lead to decomposition products which can be readily handled on an industrial scale and trigger no secondary reactions, so as to provide a more economical process for preparing high-purity trimethylolpropane.

We have found that this object is achieved by a process for removing trialkylammonium formate from methylolalkanes which have been obtained by condensation of formaldehyde with a higher aldehyde, which process comprises decomposing trialkylammonium formate at elevated temperature in the presence of a hydrogen-containing gas over a catalyst comprising ruthenium supported on titanium dioxide.

Methylolalkanes which can be worked up by the process of the present invention are, for example, neopentyl glycol, pentaerythritol, trimethylolpropane, trimethylolbutane, trimethylolethane, 2-ethyl-1,3-propanediol, 2-methyl-1,3-propane-diol, glycerol, dimethylolpropane, dipentaerythritol and 1,1-, 1,2-, 1,3- and 1,4-cyclohexane-dimethanol.

In the process of the present invention, preference is given to removing, under hydrogenating conditions, trialkylammonium formates from trimethylolalkanes which have been prepared by the organic Cannizzaro process or the hydrogenation process. Preference is given to purifying trimethylolalkanes, particularly preferably trimethylolpropane, hereinafter referred to as TMP for short, prepared by the hydrogenation process.

The preparation of crude TMP containing trialkylammonium formate by the Cannizzaro process is disclosed, for example, in DE 198 48 569.

In the hydrogenation process, the TMP is obtained by condensation of n-butyraldehyde with formaldehyde in the presence of catalytic amounts of a tertiary amine and subsequent catalytic hydrogenation of the dimethylolbutanal mixture formed. This crude TMP does not contain any alkali metal or alkaline earth metal formates or other impurities which are formed in the inorganic Cannizzaro process. Likewise, the crude TMP contains only small amounts, from about 5 to 10 mol %, of trialkylammonium formates or free trialkylamine, unlike the product obtained from the organic Cannizzaro process.

The crude TMP which comes from the hydrogenation and is to be subjected to the purification process of the present invention comprises trimethylolpropane and water together with methanol, trialkylamine, trialkylammonium formate, longer-chain linear and branched alcohols and diols, for example methylbutanol or ethylpropanediol, addition products of formaldehyde and methanol onto trimethylolpropane, acetals such as dimethylolbutyraldehyde TMP acetal and di-TMP.

Good results are obtained using crude hydrogenation products comprising from 10 to 40% by weight of trimethylolpropane, from 0 to 10% by weight of 2,2-dimethylolbutanal, from 0.5 to 5% by weight of methanol, from 0 to 6% by weight of methylbutanol, from 1 to 10% by weight of trialkylammonium formate, from 0 to 5% by weight of 2-ethyl-propanediol, from 0.1 to 10% by weight of high boilers such as di-TMP or other addition products and from 5 to 80% by weight of water. Crude hydrogenation products having such a composition can be obtained, for example, by the process described in WO 98/28253. Before the purification of the present invention to decompose the trialkylammonium formate, the crude hydrogenation product can firstly be worked up by continuous distillation as described in examples 2 and 3 of DE-A-199 63 435. However, the purification according to the present invention of the crude hydrogenation products is preferably carried out without prior treatment by distillation.

The present invention further provides a catalyst comprising ruthenium supported on shaped titanium dioxide bodies obtained by treatment of commercial titanium dioxide, before or after shaping, with from 0.1 to 30% by weight of an acid in which titanium dioxide is sparingly soluble, which catalyst is used in the process of the present invention. Ruthenium can be used either in the form of the pure metal or as a compound thereof, for example an oxide or sulfide.

The catalytically active ruthenium is applied by methods known per se, preferably to prefabricated TiO₂ as support material.

A titanium dioxide support preferred for use in the ruthenium-containing catalyst can be obtained as described in DE 197 38 464 by treatment of commercial titanium dioxide, before or after shaping, with from 0.1 to 30% by weight, based on titanium dioxide, of an acid in which the titanium dioxide is sparingly soluble. Preference is given to using titanium dioxide in the anatase modification. Examples of suitable acids are formic acid, phosphoric acid, nitric acid, acetic acid and stearic acid.

The active component ruthenium can be applied in the form of a ruthenium salt solution to the titanium dioxide support obtained in this way, using one or more impregnation steps. The impregnated support is subsequently dried and, if desired, calcined. However, it is also possible to precipitate ruthenium from a ruthenium salt solution, preferably by means of sodium carbonate, onto a titanium dioxide present as a powder in aqueous suspension. The precipitates are washed, dried, if desired calcined and shaped. Furthermore, volatile ruthenium compounds, for example ruthenium acetyl-acetonate or ruthenium carbonyl, can be brought into the gas phase and applied to the support in a manner known per se (chemical vapor deposition).

The supported catalysts obtained in this way can be in all known finished forms. Examples are extrudates, pellets or granules. Before use, the ruthenium catalyst precursors are reduced by treatment with a hydrogen-containing gas, preferably at above 100° C. The catalysts are preferably passivated by means of oxygen-containing gas mixtures, preferably air/nitrogen mixtures, at from 0 to 50° C., preferably at room temperature, before they are used in the process of the present invention. It is also possible to install the catalyst in oxidic form in the hydrogenation reactor and to reduce it under reaction conditions.

The catalyst of the present invention has a ruthenium content of from 0.1 to 10% by weight, preferably from 2 to 6% by weight, based on the total weight of the catalyst comprising catalytically active metal and support. The catalyst of the present invention can have a sulfur content of from 0.01 to 1% by weight, based on the total weight of the catalyst, with the sulfur determination being carried out coulometrically.

The ruthenium surface area is from 1 to 20 m²/g, preferably from 5 to 15 m²/g, and the BET surface area (determined in accordance with DIN 66 131) is from 5 to 500 m²/g, preferably from 50 to 200 m²/g.

The catalysts of the present invention have a pore volume of from 0.1 to 1 ml/g. Furthermore, the catalysts have a cutting hardness of from 1 to 100 N.

The above-described ruthenium-containing supported catalyst on titanium dioxide which is used according to the present invention for the decomposition of the trialkylammonium formate present in the crude TMP is also suitable for hydrogenation of the precursor of TMP (2,2-dimethylolbutanal).

The use of the same catalyst for the hydrogenation of dimethylolbutanal and for the decomposition of the trialkylammonium formate is particularly economical, since the decomposition of the trialkylammonium formate can in this case be carried out in the hydrogenation reactor of the hydrogenation process described in WO 98/28253 and no additional reactor is necessary. However, the decomposition of the trialkylammonium formates by the process of the present invention can likewise be carried out in a separate reactor.

In the process of the present invention, the decomposition of the trialkylammonium formates is generally carried out at from 100 to 250° C., preferably from 120 to 180° C. The pressures used are generally above 1×10⁶ Pa, preferably in the range from 2×10⁶ to 15×10⁶ Pa.

The process of the present invention can be carried out either continuously or batchwise, with preference being given to a continuous process.

In a continuous process, the amount of crude trimethylolalkane from the hydrogenation process or the organic Cannizzaro process is preferably from about 0.05 to about 3 kg per liter of catalyst per hour, more preferably from about 0.1 to about 1 kg per liter of catalyst per hour.

The process of the present invention is carried out under hydrogenating conditions, i.e. using an added hydrogenation gas from an external source.

As hydrogenation gases, it is possible to use any gases which comprise free hydrogen and do not contain harmful amounts of catalyst poisons, for example CO. For example, it is possible to use offgases from a reformer. Preference is given to using pure hydrogen.

The process of the present invention is illustrated by the examples below.

### EXAMPLES

I. Preparation of crude TMP by the Method of WO 98/28 253

An apparatus comprising two heatable stirred vessels connected to one another by means of overflow pipes and having a total capacity of 72 l was supplied with fresh aqueous formaldehyde solution (4 300 g/l in the form of a 40% strength aqueous solution) and n-butyraldehyde (1 800 g/h) and with fresh trimethylamine as catalyst (130 g/h) in the form of a 45% strength aqueous solution. The reactors were maintained at 40° C.

The output was fed directly into the upper part of a falling film evaporator with superposed column (11 bar steam for heating) and fractionally distilled there under atmospheric pressure to give a low-boiling top product consisting essentially of n-butyraldehyde, ethyl acrolein, formaldehyde, water and trimethylamine and a high-boiling bottom product

The top product was condensed continuously and recirculated to the above-described reactors.

The high-boiling bottom product from the evaporator (about 33.5 kg/h) was admixed continuously with fresh trimethylamine catalyst (50 g/h, in the form of a 45% strength aqueous solution) and introduced into a heatable tube reactor which was provided with random packing and had an empty volume of 12 l. The reactor was maintained at 40° C.

The output from the after-reactor was introduced continuously into the upper part of a further distillation apparatus, viz. the formaldehyde removal (11 bar steam for heating), and fractionally distilled there to give a low-boiling top product consisting essentially of ethyl acrolein, formaldehyde, water and trimethylamine and a high-boiling bottom product. The low-boiling top product (27 kg/h) was condensed continuously and recirculated to the first stirred vessel, while the high-boiling bottom product was collected.

The bottom product obtained in this way consisted essentially of water together with dimethylol butyraldehyde, formaldehyde and traces of monomethylol butyraldehyde. It was then subjected to a continuous hydrogenation. For this purpose, the reaction solution was hydrogenated at 90 bar and 115° C. in a main reactor operated in the circulation/downflow mode and a downstream after-reactor operated in the circulation mode. The catalyst was prepared by a method analogous to catalyst J in DE 198 09 418. It comprises 40% of CuO, 20% of Cu and 40% of TiO₂. The apparatus used comprised a 10 m long heated main reactor (internal diameter: 27 mm) and a 5.3 m long heated after-reactor (internal diameter: 25 mm). The flow around the circuit was 25 l/h of liquid, and the feed to the reactor was set to 4 kg/h. Accordingly, 4 kg/h of hydrogenation product were taken off. The hydrogenation product had the following composition: 22.6% by weight of TMP, 1.93% by weight of dimethylolbutanal, 1.4% by weight of methanol, 1.1% by weight of methylbutanol, 0.7% by weight of ethyl-propanediol, 1.2% by weight of adducts of TMP with formaldehyde and methanol, <0.1% by weight of TMP formate, 1.2% by weight of TMP-dimethylbutanal acetals, 2.9% by weight of high boilers, 0.57% by weight of trimethylammonium formate and 66.2% by weight of water.

II. Measurement of the Porosity

The porosity of the catalysts was determined by the Hg intrusion method corresponding to DIN 66 133.

III. Determination of the BET Surface Area

The BET surface area of the catalysts was determined in accordance with DIN 66 131.

IV. Determination of the Cutting Hardness

To determine the cutting hardness, specimens were parted by means of a cutter. The force which has to be applied to the cutter in order to cut through the specimen is the cutting hardness in N (newton).

V. Determination of the Formate Content by Means of Ion Chromatography

The formate content was determined by means of ion chromatography in accordance with DEV ISO 10304-2.

### Example 1

### Preparation of Ru/TiO₂ Catalyst

121.3 g of a ruthenium nitrosyl nitrate solution (Ru content: 10.85% by weight) were diluted with water to 90 ml. 250 g of titanium dioxide extrudates in the form of 1.5 mm extrudates having a BET surface area of 104 m²/g and a porosity of 0.36 ml/g, which had been produced as described in DE 197 38 463, example 3, were impregnated slowly with the ruthenium solution. The moist extrudates were subsequently dried at 100° C. for 2 hours and at 120° C. for 16 hours. The catalyst was activated by reduction using 10 standard l/h of hydrogen and 10 standard l/h of nitrogen at 300° C. for a period of 4 hours. The catalyst was subsequently passivated by means of air/nitrogen mixtures at room temperature.

The finished catalyst extrudates had an Ru content of 4.2% by weight, a BET surface area of 103 m²/g, a pore volume of 0.26 ml/g, a ruthenium surface area of 12 m²/g and a cutting hardness of 21.2 N.

### Examples 1 to 4

The TMP used, prepared as described above, has the composition 22.6% by weight of TMP, 1.93% by weight of dimethylolbutanal, 1.4% by weight of methanol, 1.1% by weight of methylbutanol, 0.7% by weight of ethylpropanediol, 1.2% by weight of adducts of TMP with formaldehyde and methanol, <0.1% by weight of TMP formate, 1.2% by weight of TMP dimethylbutanal acetals, 2.9% by weight of high boilers, 0.57% by weight of trimethylammonium formate and 66.2% by weight of water. 180 ml of this crude solution were treated with hydrogen at 180° C. and 90 bar in the presence of a catalyst as indicated in table 1 which had been prereduced at 180° C. and 25 bar. After one hour, the dimethylolbutanal content was determined by gas chromatography. The formate concentration was determined by means of ion chromatography. The results obtained are summarized in table 1.

| | | | Amount of | DMB³ | Formate | Formate |
|---|---|---|---|---|---|---|
| | | Shaped | catalyst | % by | % by | conversion |
| No. | Catalyst | bodies | [g] | area¹ | weight² | [%] |
| Starting material | | | | 1.93 | 0.57 | — |
| 1 | Cu/TiO₂ | 3 × 3 mm | 18.6 | <0.05 | 0.39 | 32 |
| | (DE 198 09 418) | pellets | | | | |
| 2 | Ni/SiO₂/Al₂O₃/ZrO₂ | 1.5 mm | 12.7 | <0.05 | 0.51 | 11 |
| | (EP 0672 452) | extrudates | | | | |
| 3 | Co/MnO₂/P₂O₅ | 4 mm | 21.3 | <0.05 | 0.18 | 68 |
| | (EP 0 742 045) | extrudates | | | | |
| 4 | Ru/TiO₂ | 1.5 mm | 14.8 | <0.05 | 0.006 | 99 |
| | | extrudates | | | | |

¹GC analysis (detection without water)
²Determination by means of ion chromatography
³DMB = 2,2-dimethylbutanal

It can be seen from the table that ammonium formate can be decomposed catalytically with high conversions at 150° C. over the ruthenium catalysts used according to the present invention and these catalysts are significantly more effective than copper, nickel and cobalt catalysts. Offgas analyses indicate that methane is the main product of the formate decomposition.

1. A process for removing trialkylammonium formate from methylolalkanes obtained by condensation of formaldehyde with a higher aldehyde, comprising
decomposing the trialkylammonium formate at elevated temperature, in the presence of a hydrogen-containing gas, over a catalyst comprising ruthenium supported on titanium dioxide.

2. The process of claim 1, wherein the catalyst comprises a ruthenium content of from 0.1 to 10% by weight.

3. The process of claim 1, wherein the titanium dioxide comprises shaped titanium dioxide bodies obtained by treatment of commercial titanium dioxide, before or after shaping, with from 0.1 to 30% by weight of an acid in which titanium dioxide is sparingly soluble.

4. The process of claim 1, carried out at a temperature of from 100 to 250° C.

5. The process of claim 1, carried out at a pressure of from 1×10⁶ to 15×10⁶ Pa.

6. The process of claim 1, carried out in a hydrogenation reactor.

7. A catalyst comprising ruthenium supported on shaped titanium dioxide bodies, wherein the shaped titanium dioxide bodies are obtained by treatment of commercial titanium dioxide, before or after shaping, with from 0.1 to 30% by weight of an acid in which titanium dioxide is sparingly soluble.

8. The process of claim 2, wherein the titanium dioxide comprises shaped titanium dioxide bodies obtained by treatment of commercial titanium dioxide, before or after shaping, with from 0.1 to 30% by weight of an acid in which titanium dioxide is sparingly soluble.

9. The process of claim 2, carried out at a temperature of from 100 to 250° C.

10. The process of claim 3, carried out at a temperature of from 100 to 250° C.

11. The process of claim 2, carried out at a pressure of from 1×10⁶ to 15×10⁶ Pa.

12. The process of claim 3, carried out at a pressure of from 1×10⁶ to 15×10⁶ Pa.

13. The process of claim 4, carried out at a pressure of from 1×10⁶ to 15×10⁶ Pa.

14. The process of claim 2, carried out in a hydrogenation reactor.

15. The process of claim 3, carried out in a hydrogenation reactor.

16. The process of claim 4, carried out in a hydrogenation reactor.

17. The process of claim 5, carried out in a hydrogenation reactor.

18. The process of claim 1, carried out at a temperature of from 120 to 180° C.

19. The process of claim 2, carried out at a temperature of from 120 to 180° C.

20. The process of claim 3, carried out at a temperature of from 120 to 180° C.
